(19) United States
(12) Patent Application Publication
(10) Pub. No.: US 2022/0317026 A1
(43) Pub. Date: Oct. 6, 2022

(54) FLUID TESTING SYSTEM AND APPARATUS

(71) Applicant: Combined Separation Systems Pty Ltd, Campbellfield, Victoria (AU)

(72) Inventor: Stephen PEREIRA, Campbellfield (AU)

(73) Assignee: Combined Separation Systems Pty Ltd, Campbellfield, Victoria (AU)

(21) Appl. No.: 17/638,329

(22) PCT Filed: Aug. 31, 2020
(86) PCT No.: PCT/AU2020/000096

§ 371 Date: Feb. 25, 2022

(30) Foreign Application Priority Data
Aug. 30, 2019 (AU) 2019903180

(51) Int. Cl. G01N 21/29; G01N 1/14; G01N 21/25

(52) U.S. Cl. CPC G01N 21/293; G01N 1/14; G01N 21/255; G01N 2021/1765

(57) ABSTRACT

There is disclosed a system for monitoring a state of fluid present within a fluid source comprising an apparatus configured to be mounted with respect to the fluid source, and having an inlet in fluid communication with the fluid source; a testing system mounted within the apparatus and configured to obtain a sample of fluid from the fluid source via the inlet and to perform a test of the sample of fluid; an image capture device for capturing an image of the tested sample of fluid; and a controller for controlling the operation of the apparatus so as to coordinate the collection of the sample, the testing of the sample, capture of the image, disposal of the sample and transmission of the image to a remote control centre for analysis and action.

### RELATED APPLICATIONS

The present invention claims priority from Australian provisional patent application no. 2019903180 filed on 30 Aug. 2019, the entire contents of which are incorporated herein by reference.

### FIELD OF INVENTION

The present invention relates generally to a system and method for testing a fluid sample taken from a fluid source for the presence of a predetermined condition, and in particular, to a system and method for remotely testing a fluid sample taken from a fluid source for the presence of a predetermined condition.

### BACKGROUND OF THE INVENTION

In fluid based systems such as waterways, septic tanks, sewage processing systems, waste water settlement tanks and the like, there is often a requirement to monitor the fluid contained therein for the presence of a variety of contaminants. For septic tanks and the like, such monitoring systems are important to ensure the proper and efficient working of the system to break down waste in an efficient manner. Monitoring systems enable early identification of problems associated with the fluid system such that early intervention can be achieved to keep the system in a healthy state.

For many rivers and waterways, it is important to monitor the state of the water present therein to ensure that the quality of the water is at an acceptable standard and to take appropriate action upon detection that the water is in an unacceptable state. This is particularly important where the river/waterway has numerous tributaries, and the like, that feed water into the river/waterway and which may be polluted from a variety of sources, especially after heavy rains, floods and the like. In situations where the river/waterway is used by humans, fish and other animals, there is a need to regularly update the quality of the water present therein to ensure that the water remains in an acceptable state. If toxins and pollutants are introduced into the water at a high level that could compromise the safety of humans using the water, there is a need to determine such instances so that appropriate warnings can be given to the public and the problem addressed by the appropriate authorities. In this regard, such tests are typically manually performed by authorised individuals who attend sites, such as rivers and beaches, to sample and test the water quality at regular intervals.

For operators of septic tanks and other such waste processing systems, there is also a need to monitor the state of the water and other fluids present therein, to determine the correct operation of the system. Septic tanks rely upon the presence of bacteria to process the waste entering the system resulting in the creation of clean water which can be released into the resultant soil, without having any adverse effects on the surrounding groundwater and aquifer. Solid waste is converted into sludge which falls to the bottom of the tank to break down over time. To ensure that the bacteria in the septic tank system is operating effectively and is in a healthy state, the water and sludge generated within the system is monitored to determine and assess a variety of critical performance indicators of the system. These indicators range from an assessment of the amount of ammonia, nitrate, nitrite and dissolved oxygen present within the system as well as the volume and quality of the sludge and turbidity. This may require manually taking samples of the septic tank system to test for the presence of unhealthy bacteria and where appropriate taking remedial action to rectify any identified problems. However, in situations where multiple septic tanks must be monitored and maintained over a large area, it can be very time consuming and expensive to employ multiple testing personnel to be present in the field to perform such testing and reporting. This is particularly the case for councils and the like who may manage a large number of septic tank sites over a wide area, with remote access issues, such as camping grounds, mining sites and the like.

Thus, there is a need to provide a system for conducting fluid sampling tests of a fluid system that can be performed remotely and automatically so as to capture such data at regular intervals to more closely and accurately identify problems for rectification as required.

The above references to and descriptions of prior proposals or products are not intended to be, and are not to be construed as, statements or admissions of common general knowledge in the art. In particular, the following prior art discussion does not relate to what is commonly or well known by the person skilled in the art, but assists in the understanding of the inventive step of the present invention of which the identification of pertinent prior art proposals is but one part.

### STATEMENT OF INVENTION

According to a first aspect, there is provided a system for monitoring a state of fluid present within a fluid source comprising:
an apparatus configured to be mounted with respect to the fluid source, and having an inlet in fluid communication with the fluid source;
a testing system mounted within the apparatus and configured to obtain a sample of fluid from the fluid source via the inlet and to perform a test of the sample of fluid;
an image capture device for capturing an image of the tested sample of fluid; and
a controller for controlling the operation of the apparatus so as to coordinate the collection of the sample, the testing of the sample, capture of the image, disposal of the sample and transmission of the image to a remote control centre for analysis and action.

The apparatus may comprises an enclosed box within which the testing system, image capturing device and controller may be mounted. The enclosed box may comprise an outlet in fluid communication with the fluid source through which the sample is disposed following testing.

The testing system may comprise a test tube in fluid communication with the inlet so as to receive the sample of fluid for testing. The testing system may further comprise a reagent for delivery into the test tube to facilitate mixture of the sample of fluid with the reagent to determine the presence of one or more agents in the sample of water. The reagent may be delivered into the test tube by way of a metering pump and the controller may control the operation of the metering pump to control the amount of reagent delivered into the test tube.

The testing system may further comprise a mixer, controllable by the controller, to facilitate mixing of the reagent with the sample fluid to facilitate a fluid reaction within the test tube.

The image capture device may be mounted within the apparatus so as to capture at least an image of the test tube following the fluid reaction. The image capture device may be a digital camera for taking a digital image of at least the fluid reaction. One or more lights may be mounted within the apparatus. The one or more lights may be controlled by the controller to illuminate at least the test tube for taking the digital image.

The controller may comprises a memory for storing the digital image for transmission. The controller may further comprise a transmitter for transmitting the stored image to the remote control centre for analysis and action.

In accordance with a second aspect, there is provided a method for monitoring a state of fluid present within a plurality of remote fluid sources comprising:
installing an apparatus according to the first aspect in fluid communication with each fluid source to generate test results for each fluid source at regular intervals;
receiving the test results for each fluid source;
storing the test results for each fluid source in a central database;
analysing the received test results to determine the state of the fluid present in each fluid source; and
in the event that the analysis indicates the state of fluid in one or more fluid sources is not at a predetermined standard, initiating action to address the state of fluid at the fluid source.

The test results generated for each fluid source may comprise at least a digital image of wet test of a sample of fluid taken from the fluid source and the received test results are analysed by reviewing the digital image of the wet test to determine a result of the wet test.

The result of the wet test may be determined by comparing a colour of the wet test against a predetermined colour chart.

### BRIEF DESCRIPTION OF THE DRAWINGS

The invention may be better understood from the following non-limiting description of preferred embodiments, in which:

FIG. 1 is a representation of the present invention in use in accordance with one embodiment;

FIG. 2 is a representation of the present invention in use in accordance with another embodiment;

FIG. 3 is a view of the test apparatus of the system in accordance with a preferred embodiment;

FIG. 4 shows a system of apparatus used in monitoring numerous fluid systems in accordance with the present invention; and

FIG. 5 is a flow chart depicting a method for managing testing of a variety of remote fluid sources in accordance with an embodiment of the present invention.

### DETAILED DESCRIPTION OF THE DRAWINGS

Preferred features of the present invention will now be described with particular reference to the accompanying drawings. However, it is to be understood that the features illustrated in and described with reference to the drawings are not to be construed as limiting on the scope of the invention.

The fluid test system and apparatus of the present invention will be described below in relation to its application to a septic tank system, namely for remotely checking and monitoring the health of the septic tank system. However, it will be appreciated that the fluid test system and apparatus may be employed in association with any application where there exists a body or supply of water or similar fluid for testing, as will be appreciated by those skilled in the art.

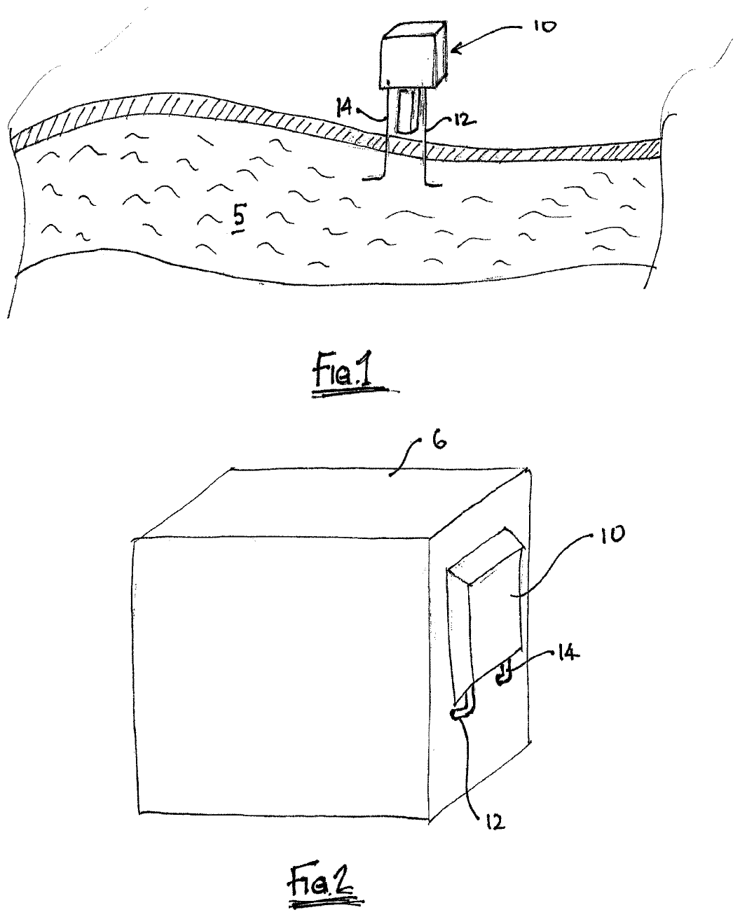

Turning to FIG. 1, the fluid test system and apparatus 10 in accordance with one embodiment of the present invention is shown in use alongside a river or beach 5 for testing the water quality present in the river or beach. In such an application, the apparatus 10 comprises an enclosed box having an inlet pipe 12 and an outlet pipe 14 extending therefrom so as to be located in the water flowing in the river or beach. In such an arrangement, the apparatus 10 is able to be remotely controlled to collect a sample of water from the river or beach by way of the inlet pipe 12, test the sample by performing tests within the enclosed box, collect the sample results and post the results for external analysis, and empty the sample back into the water via outlet pipe 14, as will be described in more detail below. The apparatus may contain a power source for independently performing he testing tasks, or may be powered by way of a renewable power source, such as a solar, wind or tidal powered system.

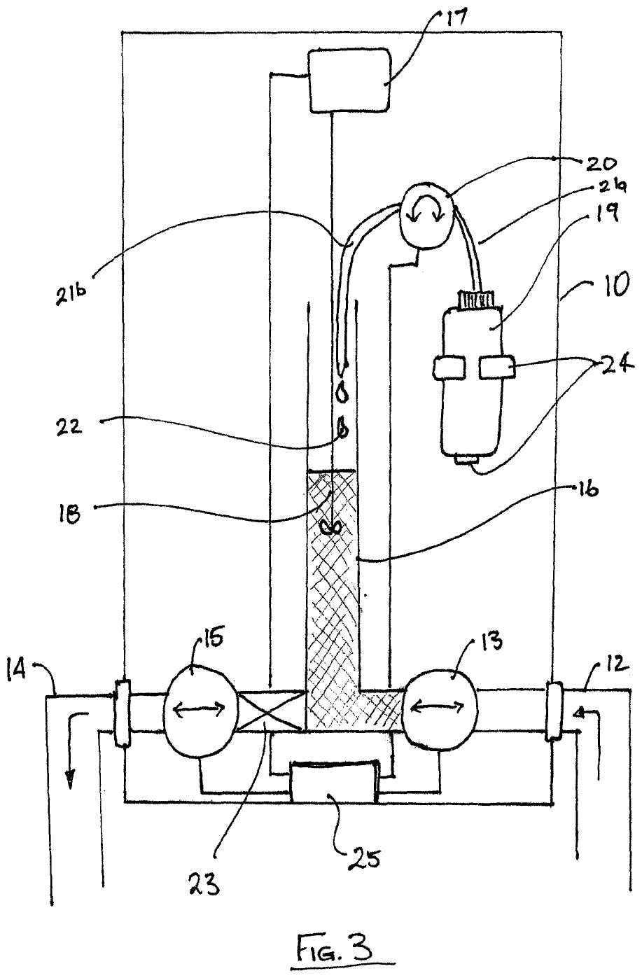

FIG. 2 depicts an alternative embodiment of the fluid test system and apparatus of the present invention. In this embodiment the apparatus 10 is mounted to a septic tank system 6 for testing the fluid within the septic tank system at any number of points within the system. The septic tank system 6 may comprise a holding tank (not shown) for receiving untreated waste and one or more secondary tanks for processing aspects of the untreated waste over time. The apparatus 10 is mounted to a wall of the septic tank system 6 and has an inlet pipe 12 for receiving a sample of fluid from a predetermined region of the septic tank system 6 and an outlet pipe 14 for disposing of the sample following testing back into the system such as that dep. It will be appreciated that whilst the apparatus is shown as being mounted on an external wall of the septic tank system 6, the apparatus 10 could be mounted on an internal wall of the septic tank system 6 or in close proximity to the septic tank system 6, and still fall within the scope of the present invention.

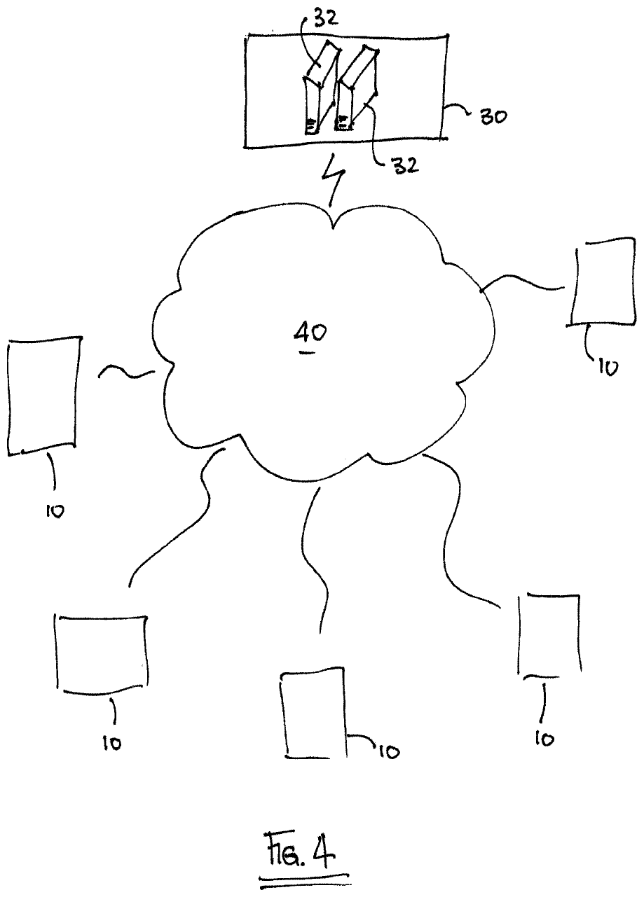

Turning to FIG. 3, an internal view of the apparatus 10 depicted in FIG. 1 and FIG. 2 is shown with the front door(s) of the apparatus removed for ease of viewing.

The inlet pipe 12 provides a means by which a sample of fluid is taken from the septic tank system 6 for testing. Pump 13 is in fluid communication with the inlet pipe 12 to create a pressure within the pipe 12 that is capable of drawing liquid from the septic tank system 6 and into a test tube 16 which extends vertically within the apparatus 10. Upon delivering a sufficient quantity of fluid into the test tube 16, the pump 13 is switched off, thereby capturing the fluid within the test tube 16 for testing. The pump 13 may be calibrated to ensure that a predetermined volume of fluid is delivered to the test tube 16 for testing.

To perform the testing function, upon a sufficient volume of fluid being present in the test tube 16, motor 20 is initiated to deliver a metered amount of a reagent 22 into the test tube 16, as depicted. The testing is typically a wet test that uses typical analytical chemistry techniques, such as PH test, Heller's test, protein test, ascorbic acid test, cloud point, and the like to indicate the presence of a specific chemical in an unknown solution. A bottle 19 containing the reagent 22 is mounted to the inside wall of the apparatus 10 by way of retainer members 24. The reagent 22 may be reagent liquid such as those used to measure the acidity or alkalinity of a solution, although other reagents are also envisaged which react and change colour upon detection of a specific condition within a fluid. A first tube 21a extends from the reagent bottle 19 to the pump 20 to deliver the reagent from the bottle 19. A second tube 21b extends from the pump 20 and into the open test tube 16 to deliver the metered amount of reagent into the test tube 16, as shown. A mixer 18 is located within the test tube 16 and as the reagent is delivered therein, the mixer 18 is activated by activating the motor 17 to rotate the mixer and mix the reagent with the fluid in the test tube 16.

After a suitable mixture time has elapsed, the mixture is allowed to stand for a reaction time after which a photograph of the test tube 16 is taken by a camera (not shown). A plurality of lights (not shown) may be mounted within the body of the apparatus 10 to illuminate the test tube for talking the photograph. The photograph is preferably taken in colour such that colormetric test depicting the true reaction is able to be captured. The camera and lights are mounted to an inner wall of the apparatus 10 immediately in front of the test tube 18. The photograph captures the reaction between the reagent 22 and the fluid, which is then sent to a remote processing station to process and determine a state of the septic tank system 6. A colour chart may be located behind the test tube 16 which is also captured by the camera when taking the photograph of the test tube to assist in colour matching the colour of the fluid present in the test tube with the appropriate colour in the chart.

Upon completion of the test, the valve 23 is opened such that the motor 15 can be activated to draw the fluid from the test tube 16 and into the outlet 14 where it can be disposed of back into the fluid source or via another disposal means. The motors may be controlled to perform a flush process or disinfection process to clean the test tube 16 and pipes of the system after each test.

It will be appreciated that a computer controller 25 is provided to control the actuation of the various features of the system. The computer controller 25 is connected via a wired or wireless connection to each pump 13, 15 and 20 so as to control the flow of fluid from the fluid source, into the test tube 16 and out of the system. The computer controller 25 may also comprise a CPU having memory that is capable of storing each of the photographs taken by the camera and for recording time data associated with each photograph. The memory may be capable of receiving and storing software for automatically controlling the sequencing and timing of the various system components. The computer controller 25 may also have a transmitter/receiver for wireless connection between all of the components of the system and for transmitting test results to a cloud storage facility or to a network for capture and analysis. Such a controller 25 enables constant remote collection and analysis of the test results for storage and upkeep.

The system as described above is also able to conduct settling tests to determine the state of any sludge or similar solid materials present in the system, as may be the case with present in the system. Similarly, as the system comprises a light controlled cabinet for generating photographs and for sending those photographs to a central remote centre for processing and monitoring, the state of the septic tank system 6 can be simply monitored and if an abnormal reading is obtained, a technician can be sent to the septic tank system for further treatment and correction. The system may also incorporate pressure and temperature gauges for measuring temperature of components of the system and the pressure of the system at various points therein. Such gauges can also be captured by the photograph and assessed at the remote location to ensure that the system is in a desirable operating condition and identify any problems in the system that may require attention.

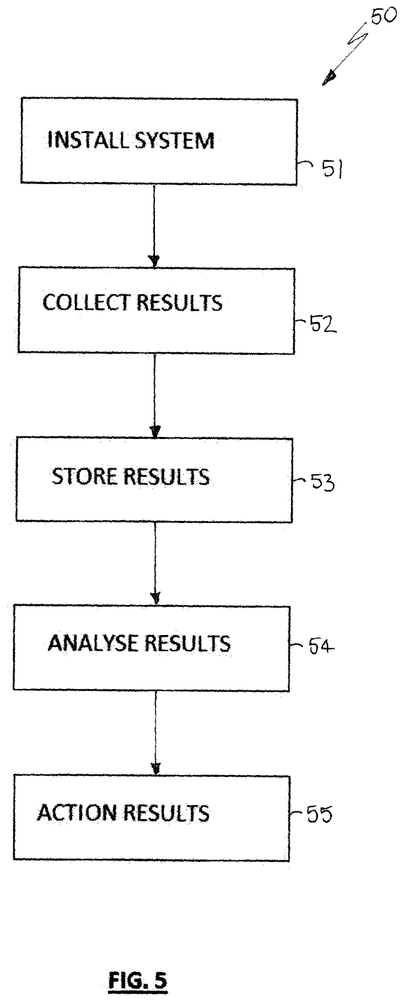

FIG. 4 depicts a system by which multiple fluid sources can be monitored and tested by a central control centre 30 using the apparatus 10 as described above. As depicted, each of the fluid sources have a testing apparatus 10 installed which is programmed by way of its computer controller 25 to take sample tests at predetermined time intervals in the manner as described above. Upon completion of each sample test, the data, including photographs, are transmitted via a distributed network 40, to be collected and processed by the control centre 30. The distributed network may be the internet or a telecommunication network. In a preferred embodiment, the computing network is the internet or a dedicated mobile or cellular network in combination with the internet, such as a GSM, CDMA, UTMS, WCDMA or LTE networks and the like. Other types of networks such as an intranet, an extranet, a virtual private network (VPN) and non-TCP/IP based networks are also envisaged.

The Control Centre 30 manages the remote apparatus in accordance with method 50 as depicted in FIG. 5.

In step 51, each remote apparatus 10 is installed at the site to be tested and is configured to conduct routine testings at predetermined intervals and to transmit a recording of the results to the control centre 30 via a remote transmission over the network. The control centre 30 then receives the test results from each apparatus 10 in step 52, ensuring that each apparatus delivers a test result and in the event that an apparatus 10 fails to deliver a test result, that apparatus 10 will be flagged by the control centre for maintenance.

In step 53, the control centre 30 stores the test results for each apparatus 10 in a server 32 and in step 54 the test sample is reviewed and analysed to determine the state of the fluid source (river/beach/septic system) associated with the apparatus 10. For photographs taken by the apparatus 10, the colour of the test mixture is able to be compared against a predetermined set of colours relating to various states of the system. The photographs can be automatically scanned by a program stored within the server 32 to match the colour of the test mixture to predetermined colours so as to determine the state of the fluid source.

In step 55, the results of the testing are actioned such that if the colour of the test mixture indicates a healthy water source, then this will be recorded against the status of the water source as stored on the server 32. However, if the colour of the test mixture indicates a problem, this will also be flagged and appropriate corrective action taken. The abnormal test result will also be stored against the water source for historical review so as to determine whether there may be other issues at play. The corrective action may be to send a maintenance team to tend to the fluid source or to initiate more regular testing to determine whether the fluid source is recovering on its own.

Further, each apparatus 10 may be provided with a CCTV system to enable remote operators to review and monitor the overall system at any time as required. Such a system enables operators to conduct complete assessments of the process and make informed decisions remotely.

It will be appreciated that the tests proposed by the apparatus as described above are able to provide an insight into the performance of various stages of the biological sewage treatment process. The results provide, with industrial standard accuracy, sufficient information for technically qualified overseers to identify issues with the process and to adjust any operating variables to maintain optimal effluent quality. The tests are able to reveal both physical properties (solids content) and chemical composition of the effluent at strategic stages of the process. The results can be provided at regular and timely intervals without the need for any expert to be located on site. Such a system contrasts significantly with other known water quality assessment systems which require on-site visits with considerably complicated equipment which prioritises “laboratory standard accuracy” over usability, regularity and/or timeliness.

When applied to biological sewage treatment plants, the system and method of the present invention provides an ability to monitor the good health of a living population of bacteria employed in the plant. Such an understanding of the population of bacteria is achievable through a full array of tests whereby the complex biochemical processes can be monitored and better understood, which is essential for managing and overseeing such processes.

It will be appreciated that the testing system of the present invention can be simply used for assessment of critical performance indicators in advanced sewage treatment plants, where the presence of ammonia, nitrate, nitrite, dissolved oxygen, sludge volume and quality and turbidity, are the key measures. However, the system and apparatus of the present invention can be readily adapted to analyse liquid samples from any number of parameters of interest, such as aquaculture, hobby aquarium suppliers, municipal water supply assessors, natural rivers and oceans. Such a system for remotely monitoring the state of a water supply offers numerus advantages to users of such systems and enables such users to take quick action to address problems as they arise.

Throughout the specification and claims the word “comprise” and its derivatives are intended to have an inclusive rather than exclusive meaning unless the contrary is expressly stated or the context requires otherwise. That is, the word “comprise” and its derivatives will be taken to indicate the inclusion of not only the listed components, steps or features that it directly references, but also other components, steps or features not specifically listed, unless the contrary is expressly stated or the context requires otherwise.

It will be appreciated by those skilled in the art that many modifications and variations may be made to the methods of the invention described herein without departing from the spirit and scope of the invention.

1. A system for monitoring a state of fluid present within a fluid source comprising:
an apparatus configured to be mounted with respect to the fluid source, and having an inlet in fluid communication with the fluid source;
a testing system mounted within the apparatus and configured to obtain a sample of fluid from the fluid source via the inlet and to perform a test of the sample of fluid;
an image capture device for capturing an image of the tested sample of fluid; and
a controller for controlling the operation of the apparatus so as to coordinate the collection of the sample, the testing of the sample, capture of the image, disposal of the sample and transmission of the image to a remote control centre for analysis and action.

2. A system according to claim 1, wherein the apparatus comprises an enclosed box within which the testing system, image capturing device and controller are mounted.

3. A system according to claim 2, wherein the enclosed box comprises an outlet in fluid communication with the fluid source through which the sample is disposed following testing.

4. A system according to claim 1, wherein the testing system comprises a test tube in fluid communication with the inlet so as to receive the sample of fluid for testing.

5. A system according to claim 4, wherein the testing system comprises a reagent for delivery into the test tube to facilitate mixture of the sample of fluid with the reagent to determine the presence of one or more agents in the sample of water.

6. A system according to claim 5, wherein the reagent is delivered into the test tube by way of a metering pump and the controller controls the operation of the metering pump to control the amount of reagent delivered into the test tube.

7. A system according to claim 6, wherein the testing system further comprises a mixer, controllable by the controller, to facilitate mixing of the reagent with the sample fluid to facilitate a fluid reaction within the test tube.

8. A system according to claim 7, wherein the image capture device is mounted within the apparatus so as to capture at least an image of the test tube following the fluid reaction.

9. A system according to claim 8, wherein the image capture device is a digital camera for taking a digital image of at least the fluid reaction.

10. A system according to claim 9, wherein one or more lights are mounted within the apparatus and are controlled by the controller to illuminate at least the test tube for taking the digital image.

11. A system according to claim 10, wherein the controller comprises a memory for storing the digital image for transmission.

12. A system according to claim 11, wherein the controller comprises a transmitter for transmitting the stored image to the remote control centre for analysis and action.

13. A method for monitoring a state of fluid present within a plurality of remote fluid sources, the method comprising:
installing an apparatus configured to be mounted with respect to the remote fluid sources, and having an inlet in fluid communication with the each fluid source to generate test results for each fluid source at regular intervals;
installing a testing system mounted within the apparatus and configured to obtain a sample of fluid from the fluid source via the inlet and to perform a test of the sample of fluid;
installing an image capture device for capturing an image of the tested sample of fluid; and
installing a controller for controlling the operation of the apparatus so as to coordinate the collection of the sample, the testing of the sample, capture of the image, disposal of the sample and transmission of the image to a remote control centre for analysis and action;
receiving the test results for each fluid source;
storing the test results for each fluid source in a central database;
analysing the received test results to determine the state of the fluid present in each fluid source; and
in the event that the analysis indicates the state of fluid in one or more fluid sources is not at a predetermined standard, initiating action to address the state of fluid at the fluid source.

14. A method according to claim 13, wherein the test results generated for each fluid source comprises at least a digital image of wet test of a sample of fluid taken from the fluid source and the received test results are analysed by reviewing the digital image of the wet test to determine a result of the wet test.

15. A method according to claim 14, wherein the result of the wet test is determined by comparing a colour of the wet test against a predetermined colour chart.
